(19) United States
(12) Patent Application Publication
(10) Pub. No.: US 2008/0146957 A1
(43) Pub. Date: Jun. 19, 2008

(54) Deep breathing training device

(76) Inventor: Michael S. Wasnick, Poulsbo, WA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/716,208

(22) Filed: Mar. 8, 2007

Related U.S. Application Data
(63) Continuation-in-part of application No. 11/639,657, filed on Dec. 14, 2006.

(51) Int. Cl. A61B 5/08

(52) U.S. Cl. 600/534

(57) ABSTRACT

A deep breathing training device includes a band worn around the abdomen which carries a switch. The switch is connected to a human-perceptible indicator. When the wearer of the device takes a deep breath and expands their abdomen, the expansion of the belt tightens the belt and thereby activates the switch and the indicator. Conversely, if the wearer merely expands their chest without expanding the abdomen, the band is not stretched and the indicator does not activate.

### CROSS-REFERENCE TO RELATED APPLICATIONS

This is a continuation-in-part of application Ser. No. 11/639,657 filed Dec. 14, 2006, pending.

### BACKGROUND OF THE INVENTION

A. Field of the Invention

This invention relates to the medical arts and more particularly to a training device for assisting a person in performing deep breathing exercises.

B. Description of Related Art

The medical benefits of deep breathing (also sometimes referred to as abdominal breathing, or diaphragmatic breathing, and belly breathing) are widely reported in the popular and medical literature. For example, Drs. Michael Roizen and Mehmet Oz report in their book You: The Owners Manual, An Insider's Guide to the Body That Will Make You Healthier and Younger (2005) that deep breathing helps transport nitric oxide, a very potent lung and blood vessel dilator that resides in your nasal passages, to your lungs thereby improving lung and blood vessel function. Taking deep breaths also helps your lungs go from 90 percent saturation of oxygen to 100 saturation of oxygen. Another benefit is that it helps improve the drainage of the lymphatic system. It further helps in stress relief and improves mental and physical performance in times of tension. Id. at pp. 165-166. A recent article in AARP magazine recommends deep breathing as a relaxation technique to take your mind off arthritis joint pain and thereby ease it.

Drs. Roizen and Oz, and other experts, suggest that deep breathing exercises be conducted such that, as the lungs fill with air, the diaphragm should pull the chest cavity down and the abdomen should move away from the spine (i.e., expand) as the lungs are filled with air. The chest also widens slightly as you inhale. After you feel your lungs feel completely full, you slowly exhale to let all the air out, taking around seven seconds. If the exercise is done with one hand placed over the abdomen (as recommended by the authors), you can use your hand to both feel the expansion of the abdomen during the inhalation and to pull the abdomen toward the spine to force all the air out of the lungs at the end of the exhalation. The exercise can be done standing, sitting or lying down.

The known prior art includes several patents related to measuring breathing performance, including U.S. Pat. Nos. 5,245,991; 5,165,393; 6,893,404; 5,311,875; and 6,740,046.

### SUMMARY OF THE INVENTION

In a first aspect, a deep breathing training device is disclosed comprising a band sized and shaped for wearing by a person about a person's abdomen, a human perceptible indicator (e.g., buzzer) and a switch. The switch is carried by the band. The indicator is activated by the switch. The band and switch are constructed such that a deep breathing exercise causes the band to move relative to the switch to activate the switch and thereby activate the indicator.

In one embodiment the device further comprises a substantially rigid panel to which the switch, the indicator and a battery for the indicator are mounted. The panel is mounted to the band. The switch may take the form of a momentary switch which is spring-biased to the off-condition. Movement of the band against the switch closes the switch and causes the indicator to activate.

In another embodiment comprising a second substantially rigid member is fastened to the band proximate to the switch. The movement of the band causes the rigid member to move against the switch to activate the switch.

In another aspect, a deep breathing training device is disclosed comprising a band sized and shaped for wearing by a person about the person's abdomen, a human perceptible indicator, a battery and a switch carried by the band. The switch and band are constructed such that the switch is activated by the band during a deep breathing exercise.

In yet another aspect, a method for training a person in deep breathing is disclosed comprising the steps of placing a band about the person such that the band extends around the person's abdomen; providing a switch with the band, the switch connected to a human perceptible indicator, and activating the switch and thereby activating the indicator due to movement of the band relative to the switch during a deep breathing exercise.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a perspective view of a deep breathing training device in accordance with a presently preferred embodiment of the invention, showing a band which is sized and shaped and for wearing about the abdomen of a person using the device.

FIG. 2 is a more detailed view perspective view, partially in section, of one possible configuration of the indicator and switch which are carried by the band of FIG. 1.

FIG. 3 shows a person wearing the device of FIG. 1 in a relaxed condition before inhaling.

FIG. 4 shows the person of FIG. 3 taking a deep abdominal breath with the expansion of the abdomen causing the indicator to be activated to alert the person that the breath was properly carried out.

FIG. 5 is a more detailed view of one possible embodiment of the band, showing an adjustment feature in the band in greater detail.

FIG. 6 is a plan view of an alternative embodiment of the device.

### DETAILED DESCRIPTION

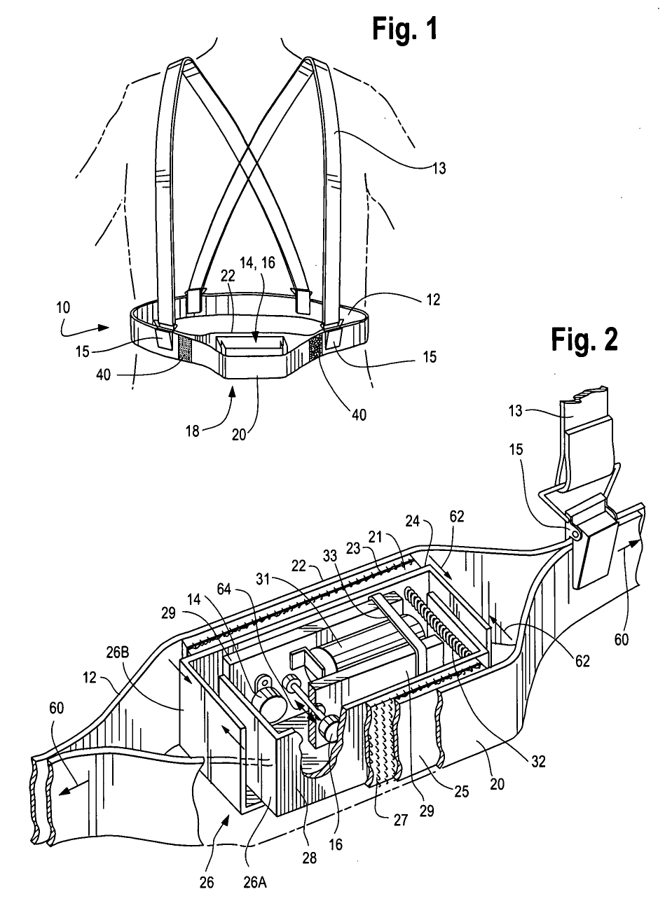

Referring now to FIG. 1, a presently preferred embodiment of a deep breathing training device 10 is shown in a perspective view. The device 10 includes a belt-like band 12 which is sized and shaped for wearing by a person about the person's abdomen. To fit a variety of sizes, the band may have a circumference of say between 24 and 50 inches and a width of say 2 to 6 inches, although these dimensions are not critical. The band may be made from any suitable material, such as cloth, leather, or other material, including an elastic material. The use of elastic material, at least in part, in the band 12 allows the band to be stretched during expansion of the abdomen during a deep breathing exercise. As will be explained below in conjunction with FIG. 2, the band includes a region 18, which may be located in the “front” of the band, i.e., adjacent to the front of the abdomen when the device is worn. The region 18 includes a front panel 20 and a rear panel 22, between which are located a human perceptible indicator 14 such as light, buzzer, vibrating element, or combination thereof, and a switch 16. The indicator 14 can also be separate from the band and connected to the band and switch by means of a wire. The indicator 14 is activated by the switch 16. In particular, during use of the device, expansion of the abdomen against the band 10 during a deep (abdominal) breathing exercise stretches or otherwise moves the band, which causes activation of the switch and thereby activation of the indicator.

The ends of the band 12 preferably include an adjustment feature 40 (such as complimentary hook and loop-type fastener) to allow the band 12 to be worn by persons of various sizes.

As best shown in FIG. 2, the indicator 14 and the switch 16 are carried by the band 10 and more particularly are sandwiched or positioned between the front and rear panels 20 and 22 of the portion 18 of the band 12. A variety of possible mounting structures can be used to carry the indicator 14 and the switch 16. In the illustrated embodiment, the switch 16 and indicator 14 are positioned within a housing 26 having a wall 24 facing the front panel 20 and a second wall 28 facing the rear portion 22. The wall 24 is attached to the front panel 20 by means of complimentary hook and loop fasteners 21 and 23 applied to the wall 24 and panel 20, respectively. Similarly, the wall 28 is attached to the rear panel 22 by means of complimentary hook and loop fasteners 27 and 25 applied to the wall 28 and panel 22, respectively.

The housing 26 is constructed so that the walls 24 and 28 can move towards each other during expansion of the band 12, indicated by the arrows 60. This expansion action (due to expansion of the abdomen during deep breathing) causes the front and rear panels 20 and 22 of the portion 18 to move towards each other as indicated by the arrows 62. This action, in turn, causes the momentary switch 16 to contact the wall 28, moving the switch as indicated by arrow 64 and overcoming the spring force biasing the momentary switch 16 to the open position, thereby closing the switch 16 and activating the indicator 14.

The housing 26 can be made from any suitable material, such as wood, cardboard, plastic, etc. The housing can be in two pieces 26A and 26B, with piece 26A simply placed on top of piece 26B. The housing can also be a one-piece construction, e.g., in the form of a housing with a bellows construction to allow the walls 24 and 28 to move towards each other. In one possible configuration, an expansion spring 32 is provided in the housing 26 biasing the housing pieces 26A and 26B away from each other so as to only allow the momentary switch 16 to contact the wall 28 when sufficient expansion of the band 12 and movement of the walls 24 and 28 toward each other has occurred. The spring constant for the spring 32 will depend on such factors as the stretchiness of the band 12, whether the entire band 12 is made from elastic material, the width of the band 12 in the region 18, the amount of force required to overcome the biasing spring in the momentary switch 16, and others, but persons skilled in the art will no doubt be able to come up with an appropriate spring. This spring 32 is not necessary, and in the embodiment of FIG. 1 there is no spring 32. The momentary switch 16 is sufficiently sensitive that only light pressure is required to activate the switch 16. Such pressure is imparted to the switch 18 by the movement of the front and rear panels 20 and 22 towards each other (indicated by arrows 62) and the resulting movement of the walls 28 and 24 of the housing 26 towards each other, causing wall 28 to come into contact with switch 16.

The device of FIG. 2 uses a 9-volt battery 31 as a power source for the indicator 14. Item 29 in FIG. 2 are spacers that space the combination of battery 31, indicator 14 and switch 16 the necessary distance between the walls 24 and 28. The spacers 29 also function as a support for the structure. One spacer 29 faces the wall 24 and other spacer 29 faces the wall 28 as shown in FIG. 2, with the switch 16 positioned on the exterior surface of one of the spacers 29. A strap 33 secures the assembly 29/31/14/16 together. The indicator 14 is wired to the switch 16 and battery 31 such than when the switch 16 closes, power is supplied to the indicator 14 and the indicator is activated. The indicator 14 in the illustrated embodiment is a simple buzzer, but other possible indicators, such as LED light, vibrating element, etc. are of course possible.

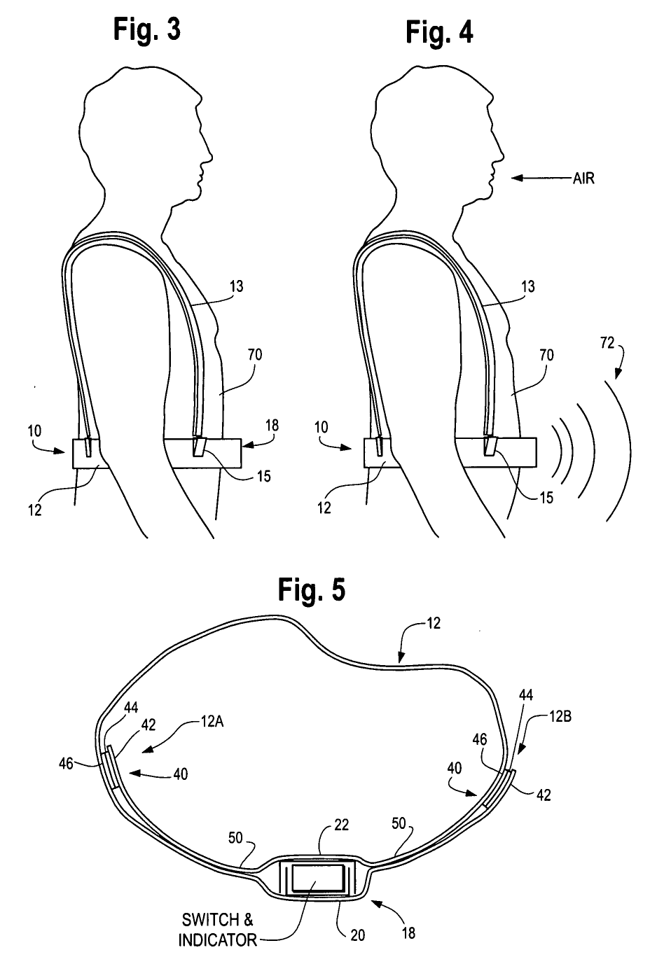

FIGS. 3 and 4 show the device 10 in use. The user places the band 12 about their waist with the suspenders 13 worn in the usual fashion. The clips 15 of the suspenders 13 are positioned on either side of the front and rear panels 20 and 22 of the elastic section 18, shown as location 50 in FIG. 5. The band at location 50 in FIG. 5 can also be sewn or fastened at this point. FIG. 3 shows the user in a relaxed condition, with the abdomen 70 in a relaxed state. FIG. 4 shows the person using the device 10 when they have filled their lungs completely in the deep breathing exercise, with the abdomen 70 extended. This extension causes the indicator to be activated indicated at 72, providing feedback to the user that the breathing exercise was properly carried out. Conversely, if they just expand their chest but with no expansion of the abdomen, then the band 12 would not be stretched/moved and the indicator 14 would typically not be activated. Accordingly, the device 10 is useful to signal to the user that the abdominal breathing exercise was properly carried out with expansion of the abdomen.

FIG. 5 is a view of one possible construction of the band 12. The band 12 is one piece and has a first end 12A and a second end 12B. The adjustment feature 40 is provided where the ends overlap the middle portion of the band. In this area, a region or strip of hook or loop fastener 44 is sewn to the band material 42 and a region or strip of complimentary hook or loop fastener 46 is sewn to the band ends 12A and 12B. These regions of hook and loop fastener allow the band to be fastened around the waist of users with different waistlines. The entire band 12 can be made out of a suitable material such as elastic, leather, cotton, nylon or other cloth, with a height of say between 2 and 6 inches.

In view of the above discussion and with reference to FIGS. 1-4, it will be appreciated that I have disclosed a method for training a person in deep breathing, comprising the steps of: placing a band 12 about the person such that the band extends around the person's abdomen 70 (FIG. 3); providing a human-perceptible indicator 14 and a switch 16 with the band (see FIG. 2), and activating the switch and thereby activating the indicator due to movement of the band during a deep breathing exercise, as shown in FIG. 4 and as discussed above. In one embodiment, as shown in FIG. 2, the switch 16 is a momentary switch which is biased to an open condition. The expansion forces on the band during the deep breathing, as indicated by the arrows in FIG. 2, overcome forces on the momentary switch 16 and cause the switch to be in a closed condition.

Note further that the deep breathing training device can be sold or furnished to users either with or without the suspenders 13. For example, if the two pieces of the band are sewn together at locations 50 in FIG. 5, the band 12 need not necessarily be used with a pair of suspenders. However, use of suspenders is optional to make sure the band 12 is placed at the correct location across the abdomen.

### Second Embodiment

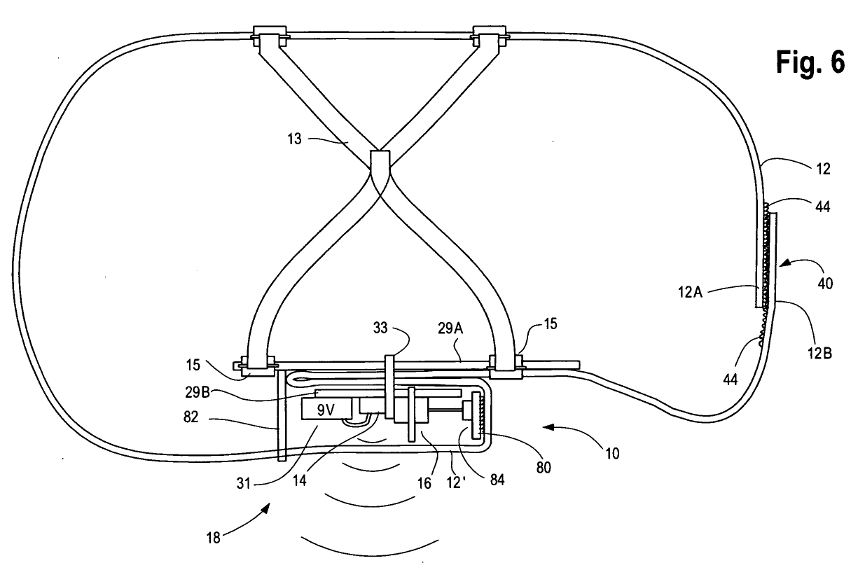

FIG. 6 is an plan view of a second embodiment of the deep breathing training device 10 which operates to alert the wearer that they have correctly performed a deep breathing exercise. The device includes a band 12 which fits about the wearer's abdomen. The band 12 includes first and second ends 12A and 12B which have complimentary hook and loop fastener 44 sewn on to the bands to provide an adjustment region 40 allowing the band to be worn by persons of different sizes. The band 12 can be made of a non-elastic cotton or nylon material, but could be made from other material including elastic materials.

The front portion 18 of the band is placed proximate to the abdomen when the device is worn. At this location there is positioned a substantially rigid support or wall 29A which is fastened to the band, e.g., using hook and loop fastener or any other convenient technique. The wall 29A is fastened to a second substantially rigid support wall 29B by means of a retaining member such as a strap or tie 33. The support wall 29B carries a battery 31, an indicator 14 (e.g., buzzer) and a momentary switch 16. A third substantially rigid member or wall 80 is fastened to the band 12 such as by using complementary hook and loop fastener or an adhesive. The third wall 80 is positioned proximate to the plunger element 84 of the momentary switch 16. The band 12 is retained in close proximity to the battery 31, indicator 14 and switch 16 by means of a ring or buckle 82.

The device 10 can be worn with or without suspenders 13. The suspenders are shown having clips 15 which are fastened to the support wall 29A and to the rear of the band as shown.

When the user places the band 12 of FIG. 6 around their abdomen, they adjust the ends 12A and 12B until the band fits comfortably about their abdomen. The wall 80 is positioned very close to the end of the switch plunger 84 and may be touching it, but does not activate the switch. However, when the wearer takes a deep breath and expands their abdomen, the expansion of the abdomen against the band tightens the band and causes the portion of the band 12′ in the region of the wall 80 to move to the left (as see in the view of FIG. 6) and thereby moves the wall 80 against the end of the plunger 84 so as to activate the switch 16. The activation of the switch 16 causes the indicator 14 to go off signaling to the user the breath was performed properly. The use of the wall 80 is optional and the device can work without it being present.

While presently preferred embodiments have been described with considerable detail, it will be appreciated that various modifications and alterations from the specifics of the disclosed embodiments are of course possible without departure from the scope of the invention. For example, the details of the construction of the indicator and the switch, the housing, the band, and how the indicator and switch are coupled to band described above are considered representative of several possible configurations but other mechanical arrangements are of course possible. All questions concerning scope of the invention are to be made with reference to the appended claims.

I claim:

1. A deep breathing training device comprising:
a band sized and shaped for wearing by a person about the person's abdomen;
a human perceptible indicator and a switch, the switch carried by the band, wherein the indicator is activated by the switch, and
wherein the band and switch are constructed such that a deep breathing exercise causes the band to move relative to the switch to activate the switch and thereby activate the indicator.

2. The device of claim 1, wherein the device further comprises a substantially rigid panel to which the switch, the indicator and a battery for the indicator are mounted, the panel mounted to the band, and wherein the switch comprises a momentary switch.

3. The device of claim 2, further comprising a second substantially rigid member fastened to the band proximate to the switch.

4. The device of claim 1, wherein the switch comprises a momentary switch.

5. The device of claim 1, further comprising suspenders connected to the band.

6. The device of claim 1, wherein the indicator is indicator is selected from the group of indicators consisting of an audible indicator, a visual indicator, and a vibrating element.

7. A deep breathing training device comprising:
a band sized and shaped for wearing by a person about the person's abdomen,
a human perceptible indicator, a battery and a switch carried by the band;
wherein the switch and band are constructed such that the switch is activated by the band during a deep breathing exercise.

8. A method for training a person in deep breathing, comprising the steps of:
placing a band about the person such that the band extends around the person's abdomen;
providing a switch with the band, the switch connected to a human perceptible indicator,
activating the switch and thereby activating the indicator due to movement of the band relative to the switch during a deep breathing exercise.

9. The method of claim 8, wherein the switch comprises a momentary switch, and wherein the movement of the band overcomes a force on the momentary switch which causes the switch to be in an open condition.

10. The method of claim 8, wherein the indicator is selected from the group of indicators consisting of an audible indicator, a visual indicator, and a vibrating element.

11. The method of claim 8, wherein the band further comprises suspenders.

12. The method of claim 8, wherein the band is made at least in part from an expandable material.

13. The method of claim 8, wherein the band further comprises a substantially rigid member fastened to the band proximate to the switch and wherein the method further comprises the step of moving the substantially rigid member against the switch to activate the indicator.
